(19) United States
(12) Patent Application Publication
(10) Pub. No.: US 2022/0068467 A1
(43) Pub. Date: Mar. 3, 2022

(54) SIMULATED FOLLOW-UP IMAGING

(71) Applicant: INTERNATIONAL BUSINESS MACHINES CORPORATION, Armonk, NY (US)

(72) Inventors: David Richmond, Newton, MA (US); Maria Victoria Sainz de Cea, Somerville, MA (US); Sun Young Park, San Diego, CA (US)

(21) Appl. No.: 17/006,931

(22) Filed: Aug. 31, 2020

(51) Int. Cl. G16H 30/40; G16H 50/50; G16H 50/20; G06T 7/00

(52) U.S. Cl. CPC G16H 30/40; G16H 50/50; G06T 2207/20081; G06T 7/0012; G16H 50/20

(57) ABSTRACT

A method, computer system, and a computer program product for simulated follow-up imaging is provided. The present invention may include receiving a set of longitudinal imaging exam data associated with a patient. The received set of longitudinal imaging exam data may correspond to a series of repeated examinations of the patient conducted over time. The present invention may also include generating, using a trained learning model, a synthetic medical image associated with the patient. The generated synthetic medical image may correspond to a simulated future imaging exam of the patient predicted based on at least a portion of the series of repeated examinations of the patient conducted over time.

### BACKGROUND

The present invention relates generally to the field of computing, and more particularly to computer-aided diagnosis.

Assessing patient risk is an inherent part of evaluating screening exams. Patients that are at higher risk of developing disease may be called back for a short term follow-up (e.g., at six months), or sent for supplemental imaging exams. Conversely, patients with abnormal findings that appear stable (e.g., low risk of malignancy), may pursue a watch-and-wait approach to treatment.

### SUMMARY

Embodiments of the present invention disclose a method, computer system, and a computer program product for simulated follow-up imaging. The present invention may include receiving a set of longitudinal imaging exam data associated with a patient. The received set of longitudinal imaging exam data may correspond to a series of repeated examinations of the patient conducted over time. The present invention may also include generating, using a trained learning model, a synthetic medical image associated with the patient. The generated synthetic medical image may correspond to a simulated future imaging exam of the patient predicted based on at least a portion of the series of repeated examinations of the patient conducted over time.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is a schematic block diagram of a medical diagnostic computer environment according to at least one embodiment;

FIG. 3 is an operational flowchart illustrating a simulated follow-up training process according to at least one embodiment;

FIG. 4 is a block diagram illustrating an exemplary simulated follow-up training process according to at least one embodiment;

FIG. 5 is an operational flowchart illustrating a simulated follow-up run-time process according to at least one embodiment;

FIG. 6 is a block diagram illustrating an exemplary simulated follow-up run-time process according to at least one embodiment;

FIG. 7 is a block diagram illustrating an exemplary a simulated current exam process according to at least one embodiment;

FIG. 8 is a block diagram illustration an exemplary patch-level simulated follow-up process according to at least one embodiment;

FIG. 9 is a block diagram of internal and external components of computers and servers depicted in FIG. 1 according to at least one embodiment;

FIG. 10 is a block diagram of an illustrative cloud computing environment including the computer system depicted in FIG. 1, in accordance with an embodiment of the present disclosure; and

FIG. 11 is a block diagram of functional layers of the illustrative cloud computing environment of FIG. 10, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, Python, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product for simulated follow-up imaging exams. As such, the present embodiment has the capacity to improve the technical field of medical imaging by automatically simulating a patient's follow-up imaging exam based on the patient's current and/or prior imaging exams. More specifically, a computer program may retrieve one or more current medical images and one or more prior medical images of a patient organized in a longitudinal order. Then, the computer program may automatically generate a synthetic medical image corresponding to a simulated future imaging exam of the patient by implementing a deep learning model to the retrieved one or more current medical images and/or one or more prior medical images of the patient.

As described previously, assessing patient risk is an inherent part of evaluating screening exams, such as, for example, breast cancer screening. Patients that are at higher risk of developing disease may be called back for a short term follow-up (e.g., at six months), or sent for supplemental imaging exams (e.g., ultrasound imaging). Conversely, patients with abnormal findings that appear stable (e.g., low risk of malignancy), may pursue a watch-and-wait approach to treatment.

Current patient risk models are based on non-imaging data, such as, the patient's and the family's history of disease. As a result, a physician, such as a radiologist, or other imaging specialist are often left with a challenging task: to integrate the output from a statistical risk model with any findings (e.g., architectural distortion; asymmetry) in a current imaging exam of the patient. These two pieces of information are not easy to integrate. For example, a radiologist may identify a mild asymmetry in a patient's mammogram, which by itself, the radiologist may not find suspicious enough to recall the patient for a short term follow-up or supplemental imaging. However, if the patient also has a 23% lifetime risk of developing breast cancer, it may be difficult for the radiologist to determine the best next steps for the patient. Furthermore, under current systems, once the patient's risk is assessed, it may not be clearly connected to a decision. For example, in places (e.g., Europe) where breast cancer screening is performed every three years—if a current imaging exam finding merits a watch-and-wait approach, it may be difficult for the radiologist to determine when to recall the patient for a follow-up exam (e.g., in six months, one year, two years).

Therefore, it may be advantageous to, among other things, provide a way to assist radiologists (or other physicians/imaging specialists) in their decision making by simulating follow-up imaging. It may also be advantageous to incorporate a patient's current and prior imaging exams, as well as other clinical information, to generate the simulated follow-up imaging. It may further be advantageous to provide physicians with a tool to enable a more intuitive way to assess patient risk and to discuss potential implications of different diagnostic planning options with the patient (e.g., treatment or watch-and-wait approach). For example, if a suspicious finding in a current medical image is predicted to change significantly over the time to the patient's next imaging exam, the patient may be advised to book a short term follow-up exam.

To address these issues and other issues, embodiments of the present disclosure propose to use machine learning of medical images to train an artificial intelligence (AI) or learning algorithm to predict follow-up imaging exams, as will be described in more detail below. According to at least one embodiment, a computer program (herein referred to as a simulated follow-up program) may retrieve a training dataset of historical imaging exam data from a database of longitudinal exams. In one embodiment, the training dataset may include a series of repeated observations (e.g., prior imaging exams) of respective patients over a time period. The training dataset may be fed into the learning algorithm to build a learning model for predicting an appearance of a future (e.g., next) imaging exam.

According to one embodiment, at run-time, the simulated follow-up program may provide the learned model with a patient's current and prior medical images. Based on the training, the learned model may process the patient's current and prior medical images and return a prediction for the appearance of a future imaging exam of the patient.

According to one embodiment, the learned model may be trained to utilize a patient's other clinical information, such as, for example, blood work, to improve the prediction of how the appearance of the future imaging exam of the patient may evolve over time. In some embodiments, additional imaging modalities (e.g., ultrasound, in the case of breast imaging) may be integrated into the learned model to improve the prediction of how benign-looking findings may evolve over time.

According to various embodiments, the above methods and systems may be applied at a patch-level of the patient's medical images. It is contemplated that focusing on the patch-level image may remove variations due to, for example, the positioning of the patient during the imaging exam. Based on a patch-level approach, a user (e.g., radiologist) may click on a region of the patient's medical image (e.g., from current exam or prior exams) and the simulated follow-up program may present the user with how that selected region is predicted to look at the next or future imaging exam. In embodiments, the patch-level approach may enable physicians to assess the risks associated with an in situ cancer identified in a patient's medical image. In some situations, the patient may live their life with no negative consequences from the identified cancer or their immune system may actually resolve the cancer naturally. By simulating future imaging procedures according to the proposed embodiments, the learned model may predict whether the in situ cancer will be stable or whether it will grow and become malignant. This determination by the simulated follow-up program may help reduce over-diagnosis and treatment of patients when a watch-and-wait approach may be more appropriate.

According to at least one embodiment of the present disclosure, the learned model may be run on all prior exams of the patient and may be used to predict the appearance of a current exam. This prediction may then be compared to an actual current imaging exam so that the radiologist may assess whether the actual current imaging exam is better (e.g., trending up) or worse (e.g., trending down) than what was predicted from the prior exams. In one embodiment, the learned model be trained on prior exams selected at different time intervals. As a result, the simulated follow-up program may generate different models corresponding to different time intervals. According to one embodiment, the different time-interval-specific models may be used by the radiologist to identify an optimal follow-up time (e.g., six months, one year, two years) by reviewing the simulated follow-up images corresponding to each time interval.

According to some embodiments, the learned model may be used to educate the patient on specific impacts on their diagnostic decisions. In other words, the learned model may be enabled to simulate the impact of a patient decision for a future imaging exam. For example, the learned model may be enabled to simulate a future follow-up exam if the patient took a specific medication and may also be enabled to simulate the future follow-up exam if the patient did not take the specific medication. In at least one embodiment, the learned model may include a generative model. As such, the learned model may be enabled to generate multiple simulated instances of the patient's future follow-up exams for review by the radiologist. Each of the multiple instances of the patient's future follow-up exams may be evaluated by another learning model to independently assess the probability of a disease (e.g., cancer) diagnosis. By evaluating multiple samples, the simulated follow-up program may generate a quantitative prediction (e.g., distribution of likelihoods) for the probability of disease in the patient's future follow-up exams. In some embodiment, the learned model may also be trained to output a predicted medical report (e.g., not just medical images) for the patient's future follow-up exam.

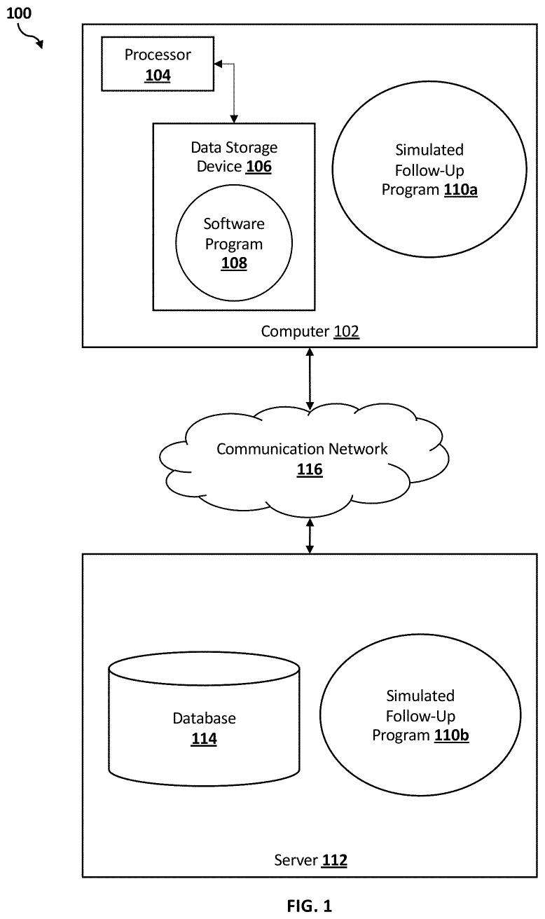

Referring to FIG. 1, an exemplary networked computer environment 100 in accordance with one embodiment is depicted. The networked computer environment 100 may include a computer 102 with a processor 104 and a data storage device 106 that is enabled to run a software program 108 and a simulated follow-up program 110a. The networked computer environment 100 may also include a server 112 that is enabled to run a simulated follow-up program 110b that may interact with a database 114 and a communication network 116. The networked computer environment 100 may include a plurality of computers 102 and servers 112, only one of which is shown. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

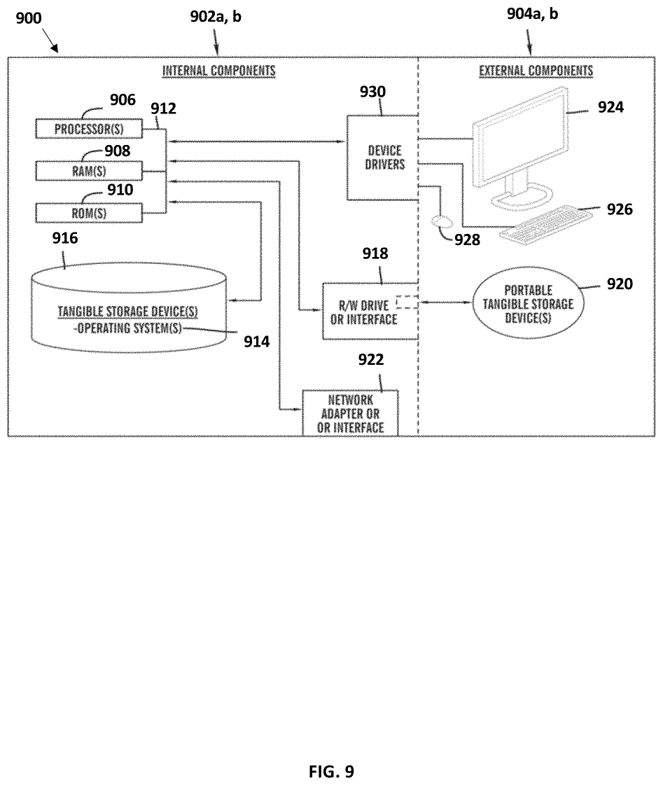

The client computer 102 may communicate with the server computer 112 via the communications network 116. The communications network 116 may include connections, such as wire, wireless communication links, or fiber optic cables. As will be discussed with reference to FIG. 9, server computer 112 may include internal components 902a and external components 904a, respectively, and client computer 102 may include internal components 902b and external components 904b, respectively. Server computer 112 may also operate in a cloud computing service model, such as Software as a Service (SaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the simulated follow-up program 110a, 110b may interact with a database 114 that may be embedded in various storage devices, such as, but not limited to a computer/mobile device 102, a networked server 112, or a cloud storage service.

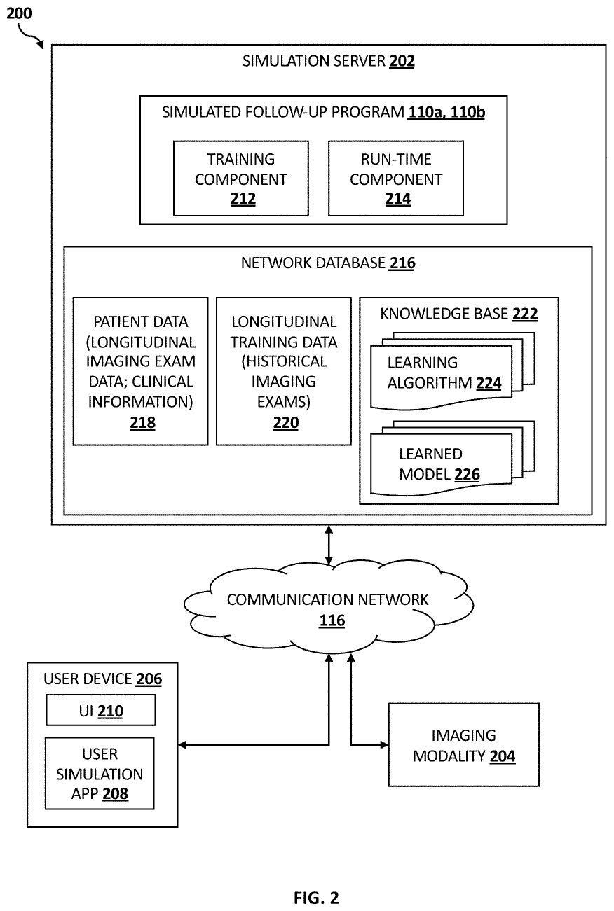

According to the present embodiment, a user using a client computer 102 or a server computer 112 may use the simulated follow-up program 110a, 110b (respectively) to train a learning algorithm to generate synthetic medical images corresponding to follow-up imaging exam (at different future time intervals or timepoints) based on longitudinal imaging exam data. The user may also utilize the simulated follow-up program 110a, 110b to generate multiple synthetic follow-up imaging exams to provide a distribution of likely outcomes for assessing patient risk. The simulated follow-up method and system are explained in more detail below with reference to FIGS. 2-8.

Referring now to FIG. 2, a schematic block diagram of a medical diagnostic computer environment 200 implementing the simulated follow-up program 110a, 110b according to at least one embodiment is depicted. Environment 200 (e.g., similar to networked computer environment 100) may include a simulation server 202, an imaging modality 204, and a user device 206, all interconnected over communication network 116 (as described in FIG. 1). Communication network 116 may include any combination of connections and protocols to support the communication between components of environment 200.

In some embodiments, the environment 200 may include fewer or additional components in various configuration that differ from the configuration illustrated in FIG. 2. For example, in some embodiments, the environment 200 may include multiple simulation servers 202 (e.g., computer systems utilizing cluster computers and components that act as a single pool of seamless resources when accessed through communication network 116), multiple imaging modalities 204, multiple user devices 205, of a combination thereof. In various embodiments, environment 200 may include one or more intermediary devices. For example, simulation server 202 may be configured to communicate with the imaging modality 204 through a gateway or separate server, such as a picture archiving and communication system (PACS) server. However, in other embodiments, the simulation server 202 may include a PACS server.

According to one embodiment, the simulation server 202 may include a computer system having a tangible storage device and a processor that is enabled to run the simulated follow-up program 110a, 110b. In one embodiment, the simulated follow-up program 110a, 110b may include a single computer program or multiple program modules or sets of instructions being executed by the processor of the simulation server 202. The simulated follow-up program 110a, 110b may include routines, objects, components, units, logic, data structures, and actions that may perform particular tasks or implement particular abstract data types. The simulated follow-up program 110a, 110b may be practiced in distributed cloud computing environments where tasks may be performed by remote processing devices which may be linked through communication network 116. In one embodiment, the simulated follow-up program 110a, 110b may include program instructions that may be collectively stored on one or more computer-readable storage media.

According to one embodiment, the imaging modality 204 may generate medical images of a patient or subject. Without any limitations, the imaging modality 204 may include a magnetic resonance image (MM) machine, an X-ray machine, a mammogram X-ray machine, an ultrasound machine, a computed tomography (CT) machine, a positron-emission tomography (PET) machine, a nuclear imaging machine, a fluoroscopy machine, an angiography machine, and any other suitable medical imaging device. The medical images generated by the imaging modality 204 may be accessible to the simulation server 202. In on embodiment, the imaging modality 204 may generate medical images and may forward (e.g., via communication network 116) the medical images to the simulation server 202. In other embodiments, the imaging modality 204 may store the generated medical images locally for subsequent retrieval or access by the simulation server 202. In various embodiments, the imaging modality 204 may transmit (e.g., via communication network 116) the generated medical images to one or more image repositories for storage (and subsequent retrieval or access by the simulation server 202. In some embodiments, one or more intermediary devices may handle images generated by the imaging modality 204. For example, the imaging modality 204 may transmit (e.g., via communication network 116) the generated images to a medical image ordering system (including information about each medical procedure), a PACS, a radiology information system (RIS), an electronic medical record (EMR), and/or a hospital information system (HIS). In one embodiment, the medical images may be formatted in the universal Digital Imaging and Communications in Medicine (DICOM) format. In one embodiment, the medical images may include embedded patient-identification labels and other tags describing the images (e.g., study type, view/laterality, study date).

According to one embodiment, the user device 206 may be, for example, a workstation, a personal computing device, a laptop computer, a desktop computer, a thin-client terminal, a tablet computer, a smart telephone, a smart watch or other smart wearable, or other electronic devices. In some embodiments, the user device 206 may be used to access images generated by the imaging modality 204, such as through the simulation server 202. In one embodiment, the simulation follow-up program 110a, 110b may include a user simulation application 208 which may be enabled to run on the user device 206 using a processor (e.g., processor 104) of the user device 206. The user simulation application 208 may include a web browser application or a dedicated device application enabled to access one or more medical images from the imaging modality 204, the simulation server 202, a separate image repository/image management system, or a combination thereof. According to one embodiment, user device 206 may also include a user interface (UI) 210. UI 210 may include human machine interfaces, such as, for example, a touchscreen, a keyboard, a cursor-control device (e.g., a mouse, a touchpad, a stylus), one or more buttons, a microphone, a speaker, and/or a display (e.g., a liquid crystal display (LCD)). For example, in some embodiments, user device 206 may include a display configured to enable graphical user interfaces (GUI) that allow a user (e.g., physician, such as a radiologist) to request a medical image, view a medical image (including a simulated medical image), manipulate a medical image, and/or generate a clinical report for one or more medical images.

According to one embodiment, the simulated follow-up program 110a, 110b may include various components, such as, for example, a training component 212 and a run-time component 214. In some embodiments, the functionality described herein as being performed by the training component 212, the run-time component 214, or both, may be distributed among multiple software components. Also, in some embodiments, the simulation server 202 may access the functionality provided by the training component 212, the run-time component 214, or both, through one or more application programming interfaces (APIs).

According to one embodiment, the simulation server 202 may include a network database 216 configured to store various data sources. In one embodiment, the network database 216 may be distributed over multiple data storage devices included in the simulation server 202, over multiple data storage devices external to the simulation server 202, or a combination thereof.

In one embodiment, the network database 216 may include a source of patient data 218 (e.g., patient database). In some embodiments, patient data 218 may be implemented in a PACS device for storing the medical images (e.g., electronic images) acquired during one or more prior imaging exams or current imaging exams using the imaging modality 204. In various embodiments, the patient data 218 may include a set of longitudinal imaging exam data based on a series of repeated observations (e.g., imaging exams) of a respective patient or subject conducted over a time period. As such, it is contemplated that longitudinal imaging exam data may be an effective approach for measuring change over time. In one embodiment, the longitudinal imaging exam data may be organized using different time intervals, such as, for example, exams performed at six months, one year, two year, or any other suitable time interval. It is contemplated that the simulated follow-up program 110a, 110b may be enabled to filter, sort, and process the longitudinal imaging exam data using different time intervals. In various embodiments, the patient data 218 may also store other clinical information regarding a patient. In some embodiments, the clinical information may include non-imaging data, such as, for example, patient's blood work results and/or family history information.

In one embodiment, the network database 216 may also include a source of training data, which may be referred to herein as longitudinal training data 220. In one embodiment, longitudinal training data 220 may be developed from historical imaging exam data configured to train a machine learning algorithm to predict future imaging exams, as described in more detail below. In one embodiment, longitudinal training data 220 may include a series of repeated observations (e.g., imaging exams) of respective patients or subjects conducted over a time period. It is contemplated that the simulated follow-up program 110a, 110b may also be enabled to filter, sort, and process the longitudinal training data 220 using different time intervals. In one embodiment, the longitudinal training data 220 may include an unannotated dataset with no ground truth labels, except for DICOM information, such as, patient-identification, study type, view/laterality, and/or study date.

According to one embodiment, the network database 216 may further include a knowledge base 222. In one embodiment, knowledge base 222 may include one or more machine learning algorithms (e.g., learning algorithm 224) and one or more trained machine learning models (e.g., learned model 226), as will be described herein. According to one embodiment, the simulated follow-up program 110, 110b may implement one or more learning algorithms 224 and one or more learned models 226 using various machine learning techniques.

Machine learning and deep machine learning (e.g., for more complex data) may generally refer to the ability of a computer program to learn without being explicitly programmed. By implementing deep machine learning techniques, the simulated follow-up program 110a, 110b may be enabled to construct one or more learned models 226 (using various learning algorithms 224) based on the example inputs in the longitudinal training data 220. According to one embodiment, the training component 212 of the simulated follow-up program 110a, 110b may build the learned models 226 using a supervised learning mechanism. Supervised learning may include feeding the learning algorithms 224 with example inputs (e.g., longitudinal training data 220) and the associated (e.g., actual) outputs (e.g., annotated or labeled data). The simulated follow-up program 110a, 110b may be configured to build the model (e.g., learned model 226) that maps the inputs to the outputs.

According to another embodiment, the training component 212 of the simulated follow-up program 110a, 110b may build the learned models 226 using an unsupervised learning mechanism. Unsupervised learning may include feeding the learning algorithms 224 with example inputs (e.g., longitudinal training data 220) which have no pre-existing outputs (e.g., unannotated or unlabeled data). In unsupervised learning, the learning algorithms 224 may be implemented to identify features and commonalities in the longitudinal training data 220 in order to extrapolate algorithmic relationships. The extrapolated algorithmic relationships may be used to build the learned models 226 configured to represent the longitudinal training data 220.

According to one embodiment, the simulated follow-up program 110a, 110b may be configured to perform deep machine learning using various types of methods and mechanisms. For example, and without limitations, the simulated follow-up program 110a, 110b may perform deep machine learning using decision tree learning, association rule learning, artificial neural networks, convolutional neural networks, recurrent neural networks, generative adversarial networks, inductive logic programming, support vector machines, clustering, Bayesian networks, reinforcement learning, representation learning, and model-based approaches. Using these approaches, the simulated follow-up program 110a, 110b may ingest, parse, and understand the longitudinal training data 220 (e.g., using training component 212) and progressively build the learned models 226 to generate (e.g., using run-time component 214) synthetic medical images which may predict and simulate follow-up imaging exams.

According to some embodiments, the knowledge base 222 may also include a source of domain-specific knowledge. In one embodiment, the knowledge base 222 may include information about one or more imaging modalities 204 including data imaging physics and artifacts and techniques used to perform various types of imaging exams or procedures (e.g., uses of contrast agents). The knowledge base 222 may also store information regarding characteristics of one or more parts of anatomy represented in the medical images produced by the various imaging modalities 204. In some embodiments, the data regarding characteristics of parts of anatomy stored in the knowledge base 222 may also be associated with patient demographic information. In various embodiments, the knowledge base 222 may include information regarding the appearance of healthy anatomical structures in medical images as well as the appearance of disease (e.g., tumors, bleeds, or other anomalies) in medical images. In yet other embodiments, the knowledge base 222 may include any other relevant medical knowledge and/or access to external sources of domain-specific knowledge.

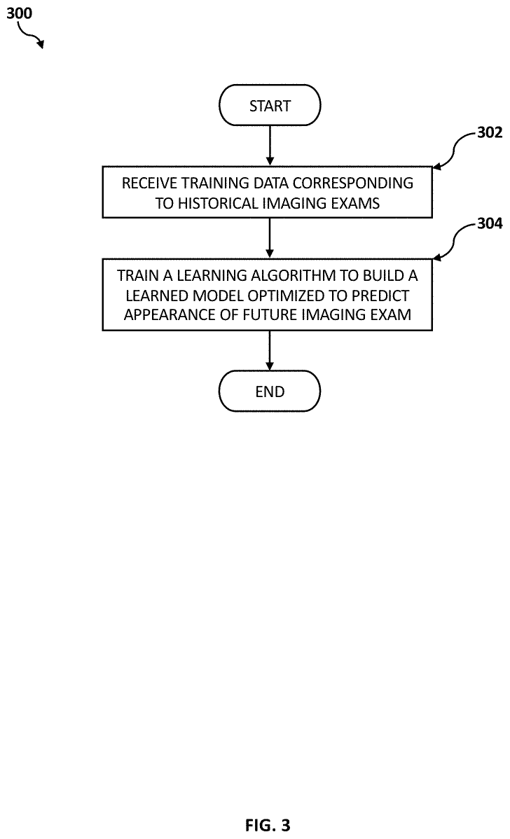

Referring now to FIG. 3, an operational flowchart illustrating an exemplary training process 300 implemented by the simulated follow-up program 110a, 110b according to at least one embodiment is depicted.

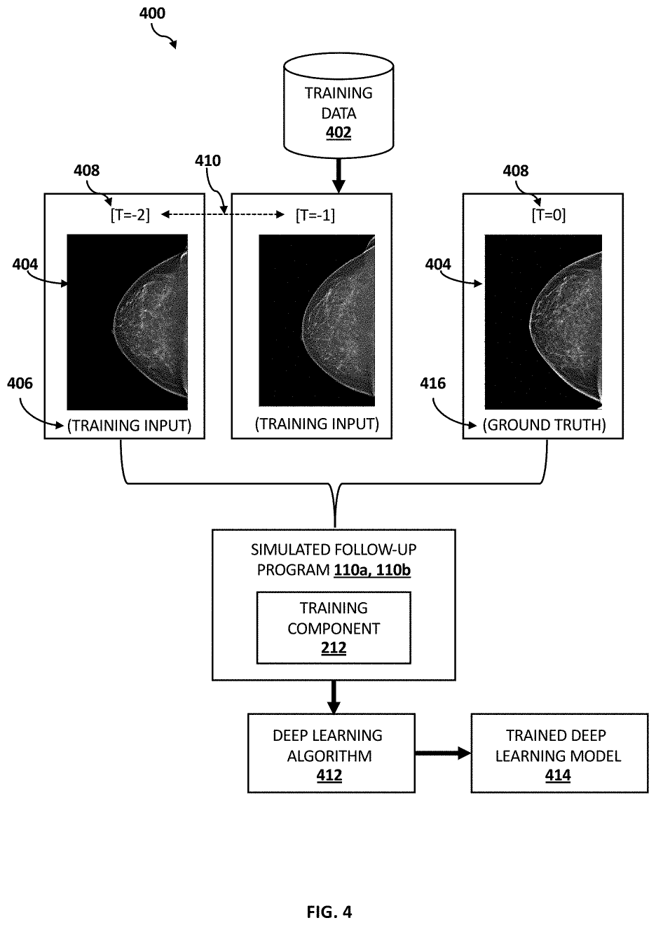

At 302, training data corresponding to historical imaging exams is received, as will be further detailed with reference to FIG. 4. In one embodiment, the training data may include longitudinal training data comprising a set of observations (e.g., imaging exams) conducted over a time period. In some embodiments, the longitudinal training data may include imaging exams conducted at different time intervals. In one embodiment, the longitudinal training data may be organized, filtered, and/or sorted according to a selected time interval (e.g., selected by the physician via user device 206).

Then at 304, a learning algorithm is trained to build a learned model that is optimized to predict an appearance of a future imaging exam, as will be further detailed with reference to FIG. 4. In one embodiment, if the longitudinal training data is filtered according to a selected time interval, the learning algorithm may be trained to build a learned model that is optimized to predict the appearance of the future imaging exam for the selected time interval. In various embodiments, the simulated follow-up program 110a, 110b may build respective learned models for different time intervals.

Referring now to FIG. 4, an exemplary block diagram illustrating a simulated follow-up training process 400 using the simulated follow-up program 110a, 110b according to at least one embodiment is depicted.

According to one embodiment, the simulated follow-up program 110a, 110b may access a source of training data 402, such as, for example, from a database of longitudinal imaging exams. In one embodiment, the simulated follow-up program 110a, 110b (e.g., training component 212) may receive one or more training inputs 404 from training data 402. Each training input 404 may include a training medical image 406 associated with historical imaging exams. Although training medical image 406 illustrates an exemplary mammography image, the simulated follow-up program 110a, 110b may be implemented with any imaging modality (e.g., imaging modality 204 as described with reference to FIG. 2. As such, training medical image 406 may include the output medical image of any imaging modality.

According to one embodiment, each training input 404 may also indicate a training time frame 408. In one embodiment, the training time frame 408 may indicate a relative time T of each training input 404 corresponding to the other training inputs 404 in the sequence of training data 402. In at least one embodiment, a training time interval 410 may be defined between successive training time frames 408 associated with the training inputs 404.

According to one embodiment, the training component 212 of the simulated follow-up program 110a, 110b may train a deep learning algorithm 412 to build a trained deep learning model 414. In some embodiments, the simulated follow-up program 110a, 110b may select one of the training inputs 404 to be a ground truth input 416. As with other training inputs 404, the ground truth input 416 may also include (as illustrated in FIG. 4) the training medical image 406 and indicate the training time frame 408.

In one embodiments, the ground truth input 416 may be configured to test the prediction accuracy of the trained deep learning model 414. For example, in one embodiment, the ground truth input 416 may be selected as a next imaging exam in a sequence of training inputs 404 in order to test the prediction accuracy of the trained deep learning model 414 with respect to the appearance of the next imaging exam. In some embodiments, in a sequence of training inputs 404 organized from a past training imaging exam (e.g., T=−2) to a present or most current training imaging exam (e.g., T=0), the most current training imaging exam (e.g., T=0) may be selected as the ground truth input 416 in order to test the prediction accuracy of the trained deep learning model 414 with respect to the appearance of the most current training imaging exam. Thereafter, the deep learning model 414 may be adjusted based on a comparison between the prediction and the ground truth input 416.

According to one embodiment, the trained deep learning model 414 may be optimized or generated for specific training time intervals 410, which may be selected by the user (e.g., radiologist). As such, multiple trained deep learning models 414 may be generated from the training data 402 corresponding to respective training time interval 410. For example, in order to predict the appearance of the next follow-up imaging exam in six months, the simulated follow-up program 110a, 110b may select the trained deep learning model 414 corresponding to the six month training time interval 410. In other words, the simulated follow-up program 110a, 110b may select the trained deep learning model 414 which was trained with the training inputs 404 spaced apart by six month training time intervals 410.

According to one embodiment, although not specifically illustrated in FIG. 4, the simulated follow-up program 110a, 110b may also train the deep learning algorithm 412 with clinical information (as described with reference to FIG. 2) to enable the trained deep learning model 414 to make more accurate predictions regarding future follow-up exams. Also, in various embodiments not specifically illustrated in FIG. 4, the simulated follow-up program 110a, 110b may train the deep learning algorithm 412 to incorporate training medical images from additional imaging modalities (e.g., incorporating both X-ray and ultrasound medical images) to enable the trained deep learning model 414 to make more accurate predictions regarding future follow-up exams. According to further embodiments not specifically illustrated in FIG. 4, the simulated follow-up program 110a, 110b may train the deep learning algorithm 412 to incorporate an impact of one or more diagnostic decisions (e.g., medical procedures, medicines) to enable the trained deep learning model 414 to make more accurate predictions regarding future follow-up exams.

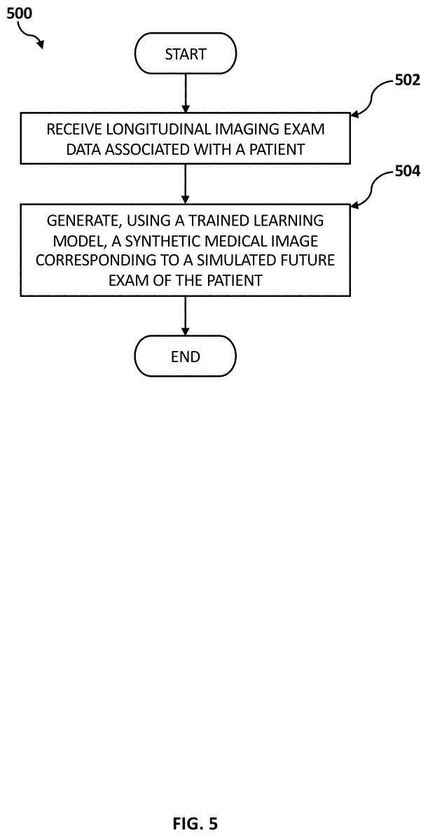

Referring now to FIG. 5, an operational flowchart illustrating an exemplary run-time process 500 implemented by the simulated follow-up program 110a, 110b according to at least one embodiment is depicted.

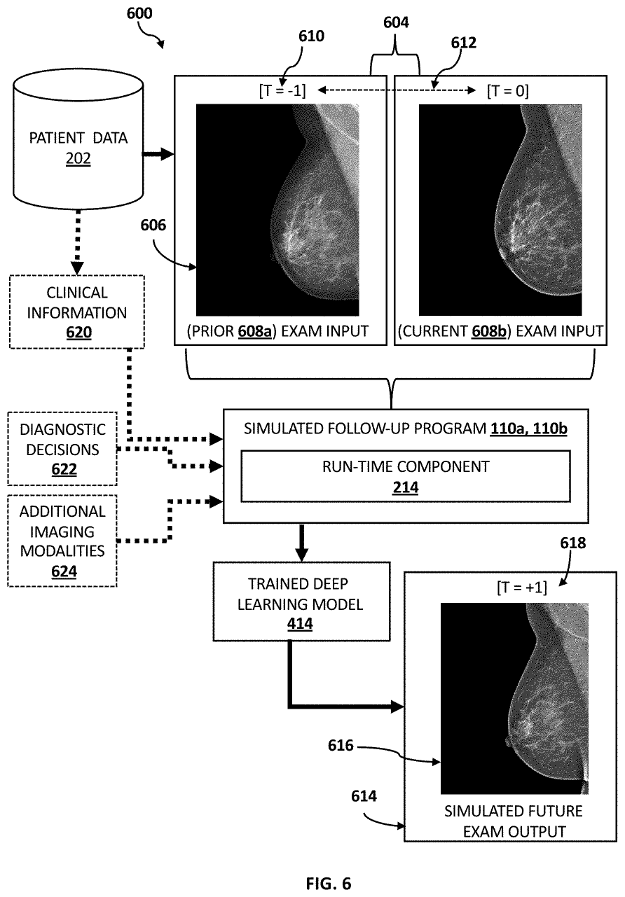

At 502, a longitudinal imaging exam data associated with a patient is received, as will be further detailed with reference to FIG. 6. In one embodiment, the received set of longitudinal imaging exam data may correspond to a series of repeated imaging exams of the patient conducted over time. In some embodiments, the received set of longitudinal imaging exam data may include a current medical image and at least one prior medical image of the patient.

Then at 504, a synthetic medical image corresponding to a simulated future imaging exam of the patient is generated using a trained learning model, as will be further detailed with reference to FIG. 6. In one embodiment, the generated synthetic medical image may be predicted based on at least a portion of the series of imaging exams of the patient conducted over time (e.g., prior exams). According to one embodiment, the generated synthetic medical image may include an image type from a same imaging modality as an input image type of the received set of longitudinal imaging exam data. For example, if the received set of longitudinal imaging exams includes ultrasound medical images, the generated synthetic medical image may also include an ultrasound medical image.

According to one embodiment, the trained learning model may receive one or more prior medical images associated with the patient and generate a synthetic medical image corresponding to a simulated current imaging exam of the patient (e.g., at the present time). According to one embodiment, the simulated follow-up program 110a, 110b may identify an actual current imaging exam of the patient and compare the actual current imaging exam with the simulated current imaging exam to determine whether the actual current imaging exam is trending up or trending down relative to what was predicted from the priors (e.g., simulated current imaging exam). In some embodiments, the simulated follow-up program 110a, 110b may display the actual current imaging exam and the simulated current imaging exam on a user device (e.g., user device 206) for diagnostic comparison by a user (e.g., radiologist).

According to one embodiment, the simulated follow-up program 110a, 110b may generate a synthetic medical image corresponding to a second or subsequent simulated future imaging exam of the patient by applying the trained learning model to at least one prior medical image of the patient, at least one current medical image of the patient, and a first simulated future imaging exam of the patient.

According to one embodiment, the simulated follow-up program 110a, 110b may implement the trained learning model as a generative model. As such, the simulated follow-up program 110a, 110b may generate multiple synthetic medical images corresponding to future follow-up imaging exams of the patient. By evaluating multiple synthetic medical images, the simulated follow-up program 110a, 110b may generate a quantitative prediction (e.g., distribution of likelihoods) for the probability of disease in the patient's future follow-up exams. In some embodiment, the trained learning model may also be trained to output a predicted medical report (e.g., not just medical images) for the patient's future follow-up exams.

According to another embodiment, the generated synthetic medical image may correspond to the simulated future imaging exam of the patient at the time interval selected by the user. In such embodiments, the simulated follow-up program 110a, 110b may determine the trained learning model to deploy based on the time interval selected by the user.

Referring now to FIG. 6, an exemplary block diagram illustrating a simulated follow-up run-time process 600 using the simulated follow-up program 110a, 110b according to at least one embodiment is depicted.

According to one embodiment, the simulated follow-up program 110a, 110b may access a source of patient data 602, such as, for example, from a database of longitudinal imaging exams. In one embodiment, the simulated follow-up program 110a, 110b (e.g., via run-time component 214) may receive one or more exam inputs 604 from patient data 602. Each exam input 604 may include a medical image 606 associated with a patient or subject. Although medical image 606 illustrates an exemplary mammography image, the simulated follow-up program 110a, 110b may be implemented with any imaging modality (e.g., imaging modality 204 as described with reference to FIG. 2. As such, medical image 606 may include the output medical image of any imaging modality. According to one embodiment, the exam inputs 604 may comprise longitudinal imaging exam data corresponding to a series of repeated imaging exams of the patient over time. In one embodiment, the exam inputs 604 may include one or more prior exam inputs 608a and one or more current exam inputs 608b. In other embodiments, the exam inputs 604 may include one or more prior exam inputs 608a and no current exam inputs 608b. In yet other embodiments, any combination of prior exam inputs 608a and current exam inputs 608b may be received by the simulated follow-up program 110a, 110b in process 600.

According to one embodiment, each exam input 604 may also indicate a time frame 610 (similar to training time frame 408 corresponding to training inputs 404). In one embodiment, the time frame 610 may indicate a relative time T of each exam input 604 corresponding to the other exam inputs 604 in the sequence of patient data 602. In at least one embodiment, a time interval 612 (similar to training time interval 410 corresponding to training inputs 404) may be defined between successive time frames 610 associated with the exam inputs 604.

According to one embodiment, the run-time component 214 of the simulated follow-up program 110a, 110b may implement the trained deep learning model 414 to generate a simulated future exam output 614. In one embodiment, the simulated future exam output 614 may include a synthetic medical image 616 generated by the trained deep learning model 414. In one embodiment, the synthetic medical image 616 may correspond to an appearance of a future follow-up imaging exam of the patient, as predicted by the trained deep learning model 414, based on at least a portion of the exam inputs 604 (e.g., series of repeated imaging exams of the patient) received by the simulated follow-up program 110a, 110b. According to at least one embodiment, the run-time component 214 of the simulated follow-up program 110a, 110b may implement the trained deep learning model 414 to generate a predicted report (e.g., natural language report) in the simulated future exam output 614 corresponding to the future follow-up imaging exam of the patient.

According to one embodiment, the simulated future exam output 614 may include a future time frame 618 associated with the future follow-up imaging exam of the patient. The future time frame 618 may indicate when the synthetic medical image 616 may correspond to the future follow-up imaging exam of the patient. For example, the future time frame 618 may indicate that the synthetic medical image 616 corresponds to the future follow-up imaging exam of the patient in six months.

As previously described, the trained deep learning model 414 may be trained using imaging exams (e.g., training inputs 404) selected at different time intervals (e.g., training time interval 410). As such, multiple trained deep learning models 414 may be generated from the training data 402 corresponding to various time intervals. According to one embodiment, the run-time component 214 of the simulated follow-up program 110a, 110b may implement the trained deep learning model 414 corresponding to the time interval 612 selected by the user (e.g., radiologist) in order to generate the synthetic medical image 616 corresponding to the future follow-up imaging exam at the time interval 612 selected by the user. In another embodiment, the simulated follow-up program 110a, 110b may generate simulated future exam outputs 614 for future follow-up imaging exam at multiple time intervals 612 (e.g., at six months, one year, two years) to assist the radiologist in identifying the optimal follow-up time for the patient (e.g., by enabling the radiologist to review the synthetic medical images 616 at each future time frame 618).

According to one embodiment, the simulated follow-up program 110a, 110b may also input clinical information 620 (as described with reference to FIG. 2) received from the patient data 602 into the trained deep learning model 414 to make more accurate predictions regarding the synthetic medical image 616 in the simulated future exam output 614. For example, a patient may have had imaging exams for breast cancer screening in the years 2016, 2017, 2018, and 2019. The patient may have also had bloodwork completed for the year 2020. The simulated follow-up program 110a, 110b may receive the exam inputs 604 for the years 2016, 2017, 2018, and 2019 and the clinical information 620 (e.g., patient bloodwork report in 2020) and generate the synthetic medical image 616 for a simulated future imaging exam in 2020. If there are no significant findings in the simulated future imaging exam output 614 for 2020, the patient and the radiologist may choose to forego the imaging exam for breast cancer screening in the year 2020.

Also, in various embodiments, the simulated follow-up program 110a, 110b may also input diagnostic decisions 622 (e.g., medications) into the trained deep learning model 414 to simulate the impact of patient diagnostic decisions on future follow-up imaging exams. According to further embodiments, the simulated follow-up program 110a, 110b may also input medical images from additional imaging modalities 624 (e.g., incorporating both X-ray and ultrasound medical images) into the deep learning model 414 to make more accurate predictions regarding the synthetic medical image 616 in the simulated future exam output 614.

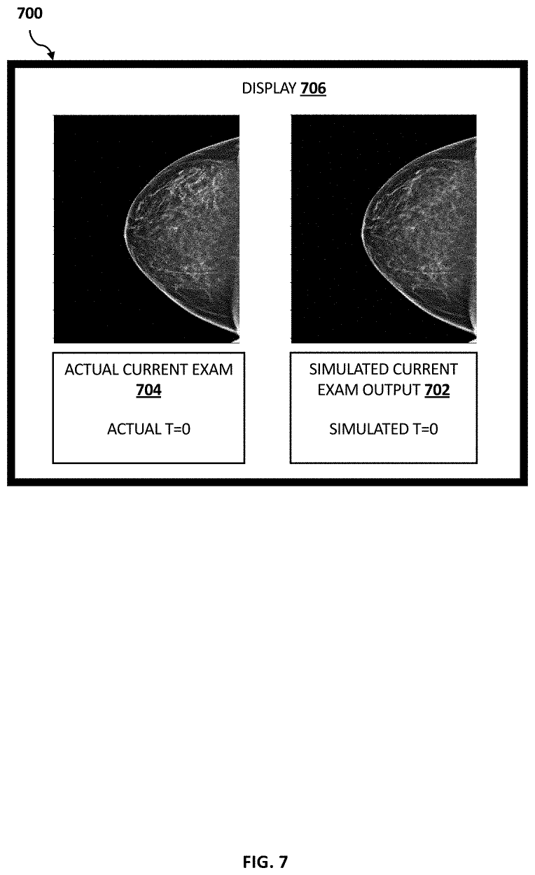

Referring now to FIG. 7, an exemplary block diagram illustrating a simulated current exam process 700 using the simulated follow-up program 110a, 110b according to at least one embodiment is depicted.

According to one embodiment, the simulated follow-up program 110a, 110b may access all prior exams of a patient (e.g., prior exam inputs 608a) to predict the appearance of a current exam. In other words, the trained deep learning model (e.g., trained deep learning model 414) may receive all prior exams of a patient (e.g., prior exam inputs 608a) and generate a simulated current exam output 702. In one embodiment, the simulated follow-up program 110a, 110b may then receive an actual current exam 704 of the patient (e.g., from patient data 602 or imaging modality 204) for comparing to the simulated current exam output 702. In one embodiment, the simulated follow-up program 110a, 110b may transmit the simulated current exam output 702 and actual current exam 704 of the patient to a display 706 of a user device for side-by-side review by a user (e.g., radiologist). In one embodiment, the side-by-side review on the display 706 may enable the user to assess whether the actual current exam 704 is trending up (e.g., better) or trending down (e.g., worse) compared to what was predicted from the priors (e.g., simulated current exam output 702).

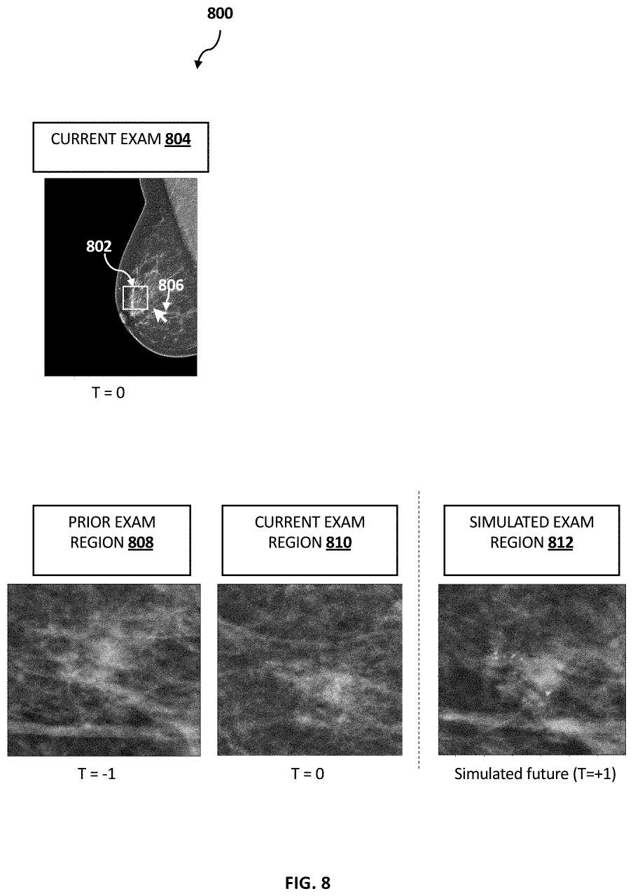

Referring now to FIG. 8, an exemplary block diagram illustrating a patch-level simulated follow-up process 800 using the simulated follow-up program 110a, 110b according to at least one embodiment is depicted.

According to one embodiment, the simulated follow-up run-time process 600 described with reference to FIG. 6 may be applied at the patch-level of patient medical images (e.g., exam inputs 604 from patient data 602) using process 800. It is contemplated that process 800 may remove variations in the appearance of simulated follow-up imaging exams due to, for example, patient positioning during the imaging exam.

According to one embodiment, the simulated follow-up program 110a, 110b may enable the user to select a region 802 of a current exam 804 using a cursor-control device 806 (e.g., a mouse, a touchpad, a stylus). In response to receiving the selection of the region 802, the simulated follow-up program 110a, 110b may feed the trained deep learning model (e.g., trained deep learning model 414) with patch-level medical images of the selected region 802 from one or more prior exams (e.g., prior exam region 808) and from the current exam (e.g., current exam region 810). According to one embodiment, the trained deep learning model may output a simulated exam region 812 to present the user with how the selected region 802 is predicted to look at a time frame (e.g., future time frame 618) associated with the future follow-up imaging exam of the patient. The patch-level approach of process 800 may be implemented to simulate the appearance of specific findings in the future to predict whether the specific finding may remain stable or automatically resolve (e.g., in which case no medical intervention may be needed).

Accordingly, the functionality of a computer may be improved by the simulated follow-up program 110a, 110b because the simulated follow-up program 110a, 110b may enable a computer to leverage a patient's current and prior imaging exams, as well as other clinical information, to generate a synthetic medical image corresponding to simulated follow-up imaging exams. The functionality of a computer may also be improved by the simulated follow-up program 110a, 110b because the simulated follow-up program 110a, 110b may enable a computer to train a deep learning model to generate synthetic future images based on longitudinal data. The functionality of a computer may further be improved by the simulated follow-up program 110a, 110b because the simulated follow-up program 110a, 110b may enable a computer train the deep learning model to generate synthetic future images at different time intervals. The functionality of a computer may additionally be improved by the simulated follow-up program 110a, 110b because the simulated follow-up program 110a, 110b may enable a computer to generate multiple synthetic follow-up exams to provide a distribution of likely outcomes for assessing risk in the patient.

It may be appreciated that FIGS. 2 to 8 provide only an illustration of one embodiment and do not imply any limitations with regard to how different embodiments may be implemented. Many modifications to the depicted embodiment(s) may be made based on design and implementation requirements.

FIG. 9 is a block diagram 900 of internal and external components of computers depicted in FIG. 1 in accordance with an illustrative embodiment of the present invention. It should be appreciated that FIG. 9 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902a, b and external components 904a, b illustrated in FIG. 9. Each of the sets of internal components 902a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the simulated follow-up program 110a in client computer 102, and the simulated follow-up program 110b in network server 112, may be stored on one or more computer-readable tangible storage devices 916 for execution by one or more processors 906 via one or more RAMs 908 (which typically include cache memory). In the embodiment illustrated in FIG. 9, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the simulated follow-up program 110a and 110b can be stored on one or more of the respective portable computer-readable tangible storage devices 920, read via the respective R/W drive or interface 918 and loaded into the respective hard drive 916.

Each set of internal components 902a, b may also include network adapters (or switch port cards) or interfaces 922 such as a TCP/IP adapter cards, wireless wi-fi interface cards, or 3G or 4G wireless interface cards or other wired or wireless communication links. The software program 108 and the simulated follow-up program 110a in client computer 102 and the simulated follow-up program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the simulated follow-up program 110a in client computer 102 and the simulated follow-up program 110b in network server computer 112 are loaded into the respective hard drive 916. The network may comprise copper wires, optical fibers, wireless transmission, routers, firewalls, switches, gateway computers and/or edge servers.

Each of the sets of external components 904a, b can include a computer display monitor 924, a keyboard 926, and a computer mouse 928. External components 904a, b can also include touch screens, virtual keyboards, touch pads, pointing devices, and other human interface devices. Each of the sets of internal components 902a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

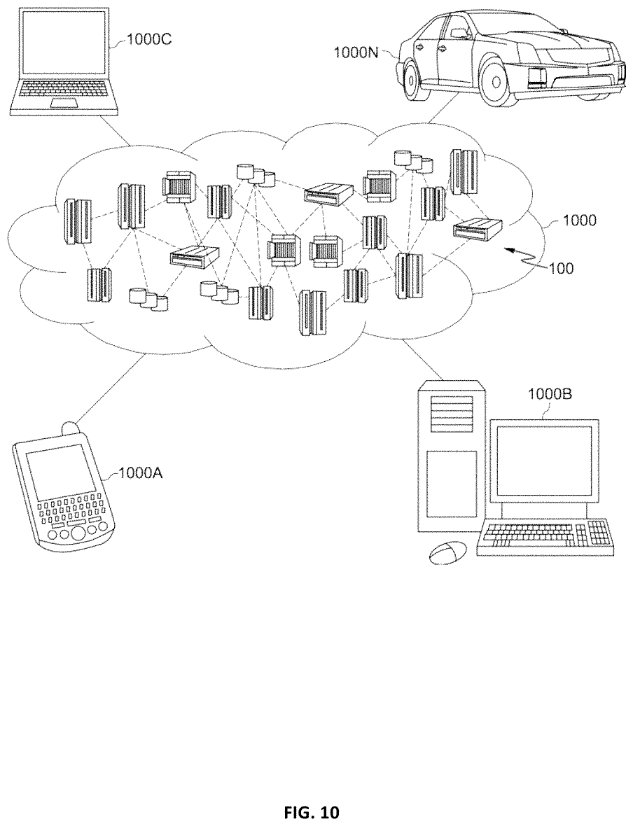

Referring now to FIG. 10, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 10 are intended to be illustrative only and that computing nodes 100 and cloud computing environment 1000 can communicate with any type of computerized device over any type of network and/or network addressable connection (e.g., using a web browser).

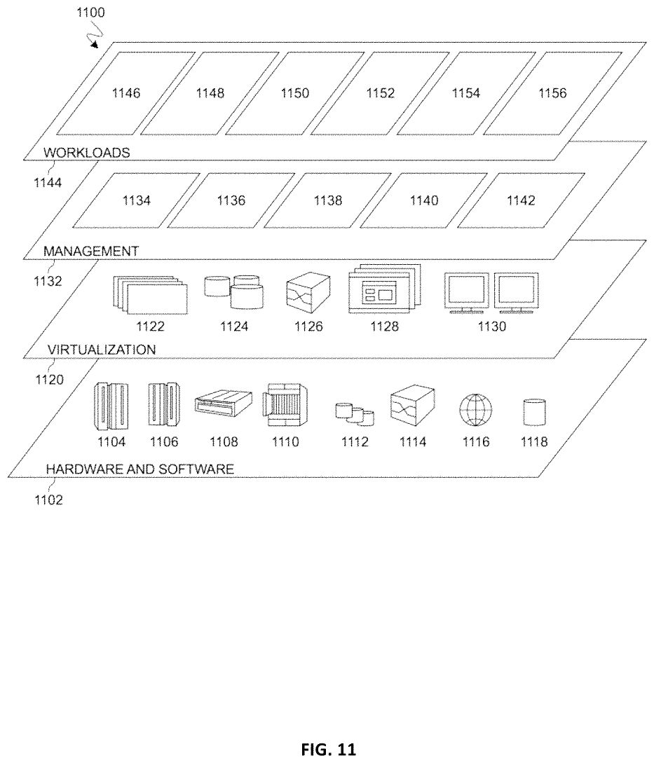

Referring now to FIG. 11, a set of functional abstraction layers 1100 provided by cloud computing environment 1000 is shown. It should be understood in advance that the components, layers, and functions shown in FIG. 11 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and simulated follow-up imaging 1156. A simulated follow-up program 110a, 110b provides a way to retrieve one or more prior medical images and current medical images of a patient organized in longitudinal order and simulate, using a deep learning model, a future imaging exam based on the prior medical images and current medical images.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method, comprising:
receiving a set of longitudinal imaging exam data associated with a patient, wherein the received set of longitudinal imaging exam data corresponds to a series of repeated examinations of the patient conducted over time; and
generating, using a trained learning model, a synthetic medical image associated with the patient, wherein the generated synthetic medical image corresponds to a simulated future imaging exam of the patient predicted based on at least a portion of the series of repeated examinations of the patient conducted over time.

2. The method of claim 1, wherein the received set of longitudinal imaging exam data includes a current medical image associated with the patient and at least one prior medical image associated with the patient.

3. The method of claim 1, further comprising:
identifying a plurality of prior medical images associated with the patient in the received set of longitudinal imaging exam data;
in response to processing the identified plurality of prior medical images, using the trained learning model, generating the synthetic medical image corresponding to the simulated future imaging exam of the patient, wherein the simulated future imaging exam includes a simulated current imaging exam of the patient;
identifying a current medical image associated with the patient in the received set of longitudinal imaging exam data, wherein the identified current medical image corresponds to an actual current exam of the patient; and
displaying the generated synthetic medical image corresponding to the simulated current exam and the identified current medical image corresponding to the actual current exam for diagnostic comparison.

4. The method of claim 1, further comprising:
receiving at least one non-imaging clinical information associated with the patient, wherein the generated synthetic medical image is based on processing the received at least one non-imaging clinical information using the trained learning model.

5. The method of claim 2, wherein the generated synthetic medical image comprises a patch-level medical image of a specific finding in the current medical image associated with the patient.

6. The method of claim 1, further comprising:
receiving a set of training data corresponding to a plurality of historical imaging examinations; and
training a learning algorithm using the received set of training data to build the trained learning model, wherein the trained learning model is optimized to predict an appearance of a future imaging exam.

7. The method of claim 6, further comprising:
filtering the received set of training data according to a selected time interval; and
training the learning algorithm using the filtered set of training data to build the trained learning model, wherein the trained learning model is optimized to predict the appearance of the future imaging exam for the selected time interval.

8. The method of claim 6, wherein the received set of training data comprises at least one different medical image from an additional imaging modality.

9. A computer system for simulated follow-up imaging, comprising:
one or more processors, one or more computer-readable memories, one or more computer-readable tangible storage media, and program instructions stored on at least one of the one or more computer-readable tangible storage media for execution by at least one of the one or more processors via at least one of the one or more memories, wherein the computer system is capable of performing a method comprising:
receiving a set of longitudinal imaging exam data associated with a patient, wherein the received set of longitudinal imaging exam data corresponds to a series of repeated examinations of the patient conducted over time; and
generating, using a trained learning model, a synthetic medical image associated with the patient, wherein the generated synthetic medical image corresponds to a simulated future imaging exam of the patient predicted based on at least a portion of the series of repeated examinations of the patient conducted over time.

10. The computer system of claim 9, wherein the received set of longitudinal imaging exam data includes a current medical image associated with the patient and at least one prior medical image associated with the patient.

11. The computer system of claim 9, further comprising:
identifying a plurality of prior medical images associated with the patient in the received set of longitudinal imaging exam data;
in response to processing the identified plurality of prior medical images, using the trained learning model, generating the synthetic medical image corresponding to the simulated future imaging exam of the patient, wherein the simulated future imaging exam includes a simulated current imaging exam of the patient;
identifying a current medical image associated with the patient in the received set of longitudinal imaging exam data, wherein the identified current medical image corresponds to an actual current exam of the patient; and
displaying the generated synthetic medical image corresponding to the simulated current exam and the identified current medical image corresponding to the actual current exam for diagnostic comparison.

12. The computer system of claim 9, further comprising:
receiving at least one non-imaging clinical information associated with the patient, wherein the generated synthetic medical image is based on processing the received at least one non-imaging clinical information using the trained learning model.

13. The computer system of claim 10, wherein the generated synthetic medical image comprises a patch-level medical image of a specific finding in the current medical image associated with the patient.

14. The computer system of claim 9, further comprising:
receiving a set of training data corresponding to a plurality of historical imaging examinations; and
training a learning algorithm using the received set of training data to build the trained learning model, wherein the trained learning model is optimized to predict an appearance of a future imaging exam.

15. The computer system of claim 14, further comprising:
filtering the received set of training data according to a selected time interval; and
training the learning algorithm using the filtered set of training data to build the trained learning model, wherein the trained learning model is optimized to predict the appearance of the future imaging exam for the selected time interval.

16. The computer system of claim 14, wherein the received set of training data comprises at least one different medical image from an additional imaging modality.

17. A computer program product for simulated follow-up imaging, comprising:
one or more computer-readable storage media and program instructions collectively stored on the one or more computer-readable storage media, the program instructions executable by a processor to cause the processor to perform a method comprising:
receiving a set of longitudinal imaging exam data associated with a patient, wherein the received set of longitudinal imaging exam data corresponds to a series of repeated examinations of the patient conducted over time; and
generating, using a trained learning model, a synthetic medical image associated with the patient, wherein the generated synthetic medical image corresponds to a simulated future imaging exam of the patient predicted based on at least a portion of the series of repeated examinations of the patient conducted over time.

18. The computer system of claim 17, wherein the received set of longitudinal imaging exam data includes a current medical image associated with the patient and at least one prior medical image associated with the patient.

19. The computer system of claim 17, further comprising:
identifying a plurality of prior medical images associated with the patient in the received set of longitudinal imaging exam data;
in response to processing the identified plurality of prior medical images, using the trained learning model, generating the synthetic medical image corresponding to the simulated future imaging exam of the patient, wherein the simulated future imaging exam includes a simulated current imaging exam of the patient;
identifying a current medical image associated with the patient in the received set of longitudinal imaging exam data, wherein the identified current medical image corresponds to an actual current exam of the patient; and
displaying the generated synthetic medical image corresponding to the simulated current exam and the identified current medical image corresponding to the actual current exam for diagnostic comparison.

20. The computer system of claim 17, further comprising:
receiving at least one non-imaging clinical information associated with the patient, wherein the generated synthetic medical image is based on processing the received at least one non-imaging clinical information using the trained learning model.
